(19) United States
(12) Patent Application Publication
(10) Pub. No.: US 2004/0053918 A1
(43) Pub. Date: Mar. 18, 2004

(54) Erythropoietin production potentiator

(75) Inventors: Shigehiko Imagawa, Tsukuba-shi (JP); Takeshi Doi, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KOWA CO., LTD., Aichi (JP)

(21) Appl. No.: 10/607,996

(22) Filed: Jun. 30, 2003

Related U.S. Application Data
(60) Provisional application No. 60/391,952, filed on Jun. 28, 2002.

(51) Int. Cl. A61K 31/551

(52) U.S. Cl. 514/218

(57) ABSTRACT

A method for treating a pathological condition caused by reduced production of erythropoietin. The method includes administering to a subject an effective amount of N,N′-bis(5(3,4,5-trimethoxyphenyl)-4-pentenyl)homopiperazine, an acid-addition salt thereof, a hydrate of N,N′-bis(5-(3,4,5-trimethoxyphenyl)-4-pentenyl)homopiperazine, or a hydrate of the salt.

### CROSS-REFERENCE TO RELATED APPLICATIONS

This application claims priority to U.S. Provisional Application No. 60/391,952, filed Jun. 28, 2002, the contents of which are hereby incorporated by reference in their entirety.

### BACKGROUND OF THE INVENTION

1. Field of the Invention

The present invention relates to an erythropoietin production potentiator, and more particularly to a method for treating pathological conditions caused by reduced production of erythropoietin, such as anemia.

2. Background of the Invention

Erythropoietin (EPO) is a glycoprotein hormone which participates in maturation and differentiation of an erythroid progenitor cell to a matured red blood cell. EPO is a 165-amino-acid polypeptide which is found in nature in the form of a monomer, Lin, F-K. et al., Proc. Natl. Acad. Sci. USA 82:7580-7584 (1985).

Human erythropoietin plays a key role in proliferation and differentiation of red blood cells. Therefore, the hormone is useful for treatment of blood diseases primarily involving reduced production of red blood cells. Clinically, EPO is used in treatment of anemia associated with chronic renal failure (CRF), autologous blood storage, or anemia of prematurity (Eschbach, J W, Egrie, J C, Downing, M R, et al., NEJM, 316:73-78 (1987); Eschbach, J W, Abdulhadi, M H, Browne, J K et al., Ann. Intern. Med., 111:992 (1989); Egrie, J C, Eschbach, J W, McGuire, T, Adamson, J W, Kidney Intl., 33:262 (1988); and Lim, V S, Degowin, R L, Zavala, D, et al., Ann. Intern. Med., 110:108-114 (1989)). EPO is also used in treatment of patients suffering AIDS or patients having cancer and receiving chemotherapy (Danna, R P, Rudnick, S A, Abels, R I, M B, Garnick, Erythropoietin in Clinical Applications—An International Perspective, New York: Marcel Dekker; 1990, pp. 301-324). EPO has been found to be effective in treatment of chronic anemia.

Some proteins used for therapy, such as EPO, have a short plasma half-life and are susceptible to degradation in the presence of protease, Spivak, J. L. and Hogans, B. B., Blood, 73:90 (1989); McMahon, F. G. et al., Blood, 76:1718 (1990); i.e., EPO exhibits poor bioavailability. Accordingly, in a protein therapy employing EPO, intravenous injection must be performed frequently in order to maintain the effective therapeutic blood level of the protein in circulation. Subcutaneous injection may be an alternative administration route. However, when administered subcutaneously, the agent is absorbed slowly from the administration site. Thus, plasma level of the protein is significantly lower as compared with the case of intravenous administration, although the effect of sustained release may be appreciable. Therefore, in order to obtain a therapeutic effect of similar level, subcutaneous injection must be performed frequently as in the case of intravenous administration.

Accordingly, demand exists for a method for potentiating erythropoietin production through administration of a compound other than erythropoietin, as EPO exhibits poor bioavailability.

### BRIEF SUMMARY OF THE INVENTION

In view of the foregoing, the present inventors have performed studies on effects of various compounds on erythropoietin production, and, quite unexpectedly, have found that N,N′-bis(5-(3,4,5-trimethoxyphenyl)-4-pentenyl)homopiperazine (compound 1), an acid-addition salt thereof, a hydrate of N,N′-bis(5-(3,4,5-trimethoxyphenyl)-4-pentenyl)homopiperazine, or a hydrate of the salt exhibits an erythropoietin production potentiating effect, and thus is useful as a preventive or therapeutic drug for anemia, thereby leading to completion of the present invention.

Accordingly, the present invention provides a method for treating pathological conditions caused by reduced production of erythropoietin, comprising administering to a subject an effective amount of N,N′-bis(5-(3,4,5-trimethoxyphenyl)-4-pentenyl)homopiperazine, an acid-addition salt thereof, a hydrate of N,N′-bis(5-(3,4,5-trimethoxyphenyl)-4-pentenyl)homopiperazine, or a hydrate of the salt.

The present invention also provides a method for potentiating erythropoietin production in a subject, comprising administering, to the subject, an effective amount of N,N′-bis(5-(3,4,5-trimethoxyphenyl)-4-pentenyl)homopiperazine, an acid-addition salt thereof, a hydrate of N,N′-bis(5-(3,4,5-trimethoxyphenyl)-4-pentenyl)homopiperazine, or a hydrate of the salt.

The present invention also provides an erythropoietin production potentiator containing, as an active ingredient, N,N′-bis(5-(3,4,5-trimethoxyphenyl)-4-pentenyl)homopiperazine, an acid-addition salt thereof, a hydrate of N,N′-bis(5-(3,4,5-trimethoxyphenyl)-4-pentenyl)homopiperazine, or a hydrate of the salt.

The present invention also provides a preventive or therapeutic agent for pathological conditions caused by reduced production of erythropoietin containing, as an active ingredient, N,N′-bis(5-(3,4,5-trimethoxyphenyl)-4-pentenyl)homopiperazine, an acid-addition salt thereof, a hydrate of N,N′-bis(5-(3,4,5-trimethoxyphenyl)-4-pentenyl)homopiperazine, or a hydrate of the salt.

The present invention also provides use of N,N′-bis(5-(3,4,5-trimethoxyphenyl)-4-pentenyl)homopiperazine, an acid-addition salt thereof, a hydrate of N,N′-bis(5-(3,4,5 trimethoxyphenyl)-4-pentenyl)homopiperazine, or a hydrate of the salt in production of an erythropoietin production potentiator.

The present invention also provides for use of N,N′-bis(5-(3,4,5-trimethoxyphenyl)-4-pentenyl)homopiperazine, an acid-addition salt thereof, a hydrate of N,N′-bis(5-(3,4,5 trimethoxyphenyl)-4-pentenyl)homopiperazine, or a hydrate of the salt in the production of a preventive or therapeutic agent for pathological conditions caused by reduced production of erythropoietin.

The invention also contemplates the administration of a N,N′-bis(5-(3,4,5-trimethoxyphenyl)-4-pentenyl)homopiperazine compound, salt or hydrate to increase red blood cell levels, platelet levels, or reduce hypoxia. Conditions associated with living at a high altitude or in hypoxic environments; high-altitude affinity hemoglobinopathy; hypoxia; smoking; chronic obstructive pulmonary disease; cyanotic heart disease, sleep apnea, renal hypoxia; and anemias associated with autoimmune diseases, chronic infections, rheumatoid arthritis, AIDS, or malignancy; may be treated with the N,N′-bis(5-(3,4,5-trimethoxyphenyl)4-pentenyl)homopiperazine compound, its salt or hydrate of the present invention.

The compounds of the present invention may also be administered along with other drugs or growth factors, such as hematopoietic growth factors, including stem cell factor (SCF), IGF-1, and IL-3, to promote the expansion and maturation of red blood cells or megakaryocytes.

The present invention also provides a diagnostic method in which a N,N′-bis(5-(3,4,5-trimethoxyphenyl)-4-pentenyl)homopiperazine compound, salt or hydrate is administered to a subject, and the rate and/or rise of the subjects EPO level is determined. Such a method reflects the ability of a subject to produce EPO. Determination that a subject has a reduced ability to produce EPO in response to administration of the compounds of the present invention, compared to a pre-disease baseline measurement in a subject or compared to a baseline established for normal subjects, would be helpful in diagnosing the presence or extent of an EPO associated disease. Similarly, an enhanced ability to release EPO in response to the compounds of the present invention would be indicative of other conditions.

### BRIEF DESCRIPTION OF THE DRAWINGS

FIG. 1 shows effect of compound 1 on the amount of EPO produced by Hep3B cells under 21% O₂;

FIG. 2 shows effect of compound 1 on the amount of EPO produced by Hep3B cells under 1% O₂; and

FIG. 3 shows effect of L-NMMA or compound 1 on EPO potentiator activity.

### DETAILED DESCRIPTION OF THE INVENTION

The active ingredient of the present invention; namely, N,N′-bis(5-(3,4,5-trimethoxyphenyl)-4-pentenyl)homopiperazine (compound 1), an acid-addition salt thereof, a hydrate of N,N′-bis(5-(3,4,5-trimethoxyphenyl)-4-pentenyl)homopiperazine, or a hydrate of the salt, is a compound that has been known to be useful as a brain protecting agent for amelioration of brain dysfunction and prevention of aggravation of brain dysfunction (Japanese Patent Application Laid-Open (kokai) No. 3-2144); an anti-allergic agent or an anti-inflammatory agent based on cell adhesion inhibitory effect or cell infiltration inhibitory effect (Japanese Patent Application Laid-Open (kokai) No. 9-143075); or a therapeutic agent for Sjogren's syndrome, conjunctival disorder, etc. based on apoptosis inhibitory effect (WO02/20477). However, nothing has been known as to what effect the above compound 1 exerts on production of erythropoietin.

Compound 1 can be produced through a method described in, for example, Japanese Patent Application Laid-Open (kokai) No. 3-2144 or Japanese Patent Application Laid-Open (kokai) No. 9-143075.

In the present invention, an acid-addition salt of compound 1, a hydrate of the compound 1, or a hydrate of the acid-addition salt may also be employed, and each of the acid-addition salt and hydrates may be obtained through a routine method. Examples of the acid which forms such an acid-addition salt include inorganic acids such as sulfuric acid, hydrochloric acid, nitric acid, phosphoric acid, and hydrobromic acid; and organic acids such as acetic acid, lactic acid, succinic acid, tartaric acid, malic acid, maleic acid, citric acid, fumaric acid, methanesulfonic acid, and toluenesulfonic acid.

Compound 1 exhibits strong erythropoietin production potentiating effect as described in the below Examples, and thus is useful as a preventive or therapeutic agent for pathological conditions of mammals, including humans, caused by reduced production of erythropoietin, such as anemia, particularly for chronic anemia, renal anemia, hypoplastic anemia, or pure blood cell aplasia.

The medical compound to be employed in the present invention may be used singly or in combination with a pharmacologically acceptable carrier such as a solubilizing agent, an excipient, a binder, or a diluent. The compound or the composition may assume a dosage form such as tablets, capsules, granules, a powder, a lotion, a plaster, an injection, a form suitable for intravenous administration, a form suitable for intramuscular or subcutaneous administration, a time-release preparation, a depot preparation, an aerosol or a suppository. These drug preparations are produced through known methods. For example, an oral drug preparation may be produced through processing the medical compound of the present invention with a solubilizing agent such as tragacanth gum, acacia, a sucrose ester, lecithin, olive oil, soybean oil, or PEG400; an excipient such as starch, mannitol, or lactose; a binder such as sodium carboxymethylcellulose or hydroxypropylcellulose; a disintegrant such as crystalline cellulose or calcium carboxymethylcellulose; a lubricant such as talc or magnesium stearate; or a flowability-improving agent such as light anhydrous silicic acid, which are suitably selected.

The drug of the present invention is administered perorally or parenterally. The dose of the drug of the present invention differs depending on the body weight, age, sex, medical conditions, etc. of the patient. The daily dose of compound 1 for an adult is typically 0.01 to 1,000 mg, preferably 0.1 to 100 mg. All intermediate values and subranges are also specifically contemplated, for instance, a daily dose of 0.02, 0.03, 0.05, 0.1, 0.2, 0.5, 1.0, 2.0, 5.0, 10.0, 15, 25, 50, 75, 100, 200, 500, 750 or 900 mg, may be used. Compound 1 is preferably administered once a day, or two or three times a day in a divided manner.

### EXAMPLES

The present invention will next be described in more detail by way of examples, which should not be construed as limiting the technical scope of the present invention thereto.

A. Materials and methods

Hep3B cells, which produce EPO at a low O₂ partial pressure, were cultured in an amount of 1.5-3.0×10⁶ cells/petri dish (100 mm) by use of Dulbecco's modified eagles' medium (DMEM)/10% fetal calf serum (FCS) (10 ml). The next day, the culture liquid of each petri dish was renewed, and a predetermined amount of compound 1 was added thereto, followed by culture for 24 hours at 37° C. under 1% O₂ or 21% O₂. EPO contained in the resultant culture supernatant was measured through ELISA. Separately, EPO potentiator activity was measured in the following manner. The luciferase-pXP2 (serving as a reporter gene) of a Hep3B wild-type gene was ligated with an enhancer and potentiator fragment (144 bp), to thereby produce a modified Hep3B wild-type gene as Pwt. Hep3B cells were cultured in an amount of 1×10⁶ cells/petri dish (30 mm) by use of 4-mL DMEM/10% FCS, and the above modified wild-type (2 μg) and β-galactosidase gene (1 μg) (serving as an internal standard) were inserted into each of the cultured cells through the lipofectin method. The next day, the culture liquid of each petri dish was renewed, and a predetermined amount of compound 1 or L-NMMA was added thereto, followed by culture for 24 hours at 37° C. under 1% O₂ or 21% O₂ (reference). After completion of culture, luciferase activity and β-galactosidase activity of the resultant cell extract were determined. EPO potentiator activity is represented by the following formula.

HI (hypoxic induction)=(1% O₂ luciferase/β-galactosidase)/(21% O₂ luciferase/β-galactosidase)

B. Results

FIG. 1 shows effect of compound 1 on the amount of EPO produced by Hep3B cells in the atmosphere (at a normal O₂ partial pressure). In control samples, the amount of EPO was found to be 33.6±8.7 mU/protein μg. In the cases where compound 1 was added in amounts of 10, 15, and 30 μM, the amount of EPO was found to be 49.5±2.3, 56.4±2.2, and 116.8±5.2 mU/protein μg, respectively; i.e., the amount of EPO increased dose-dependently.

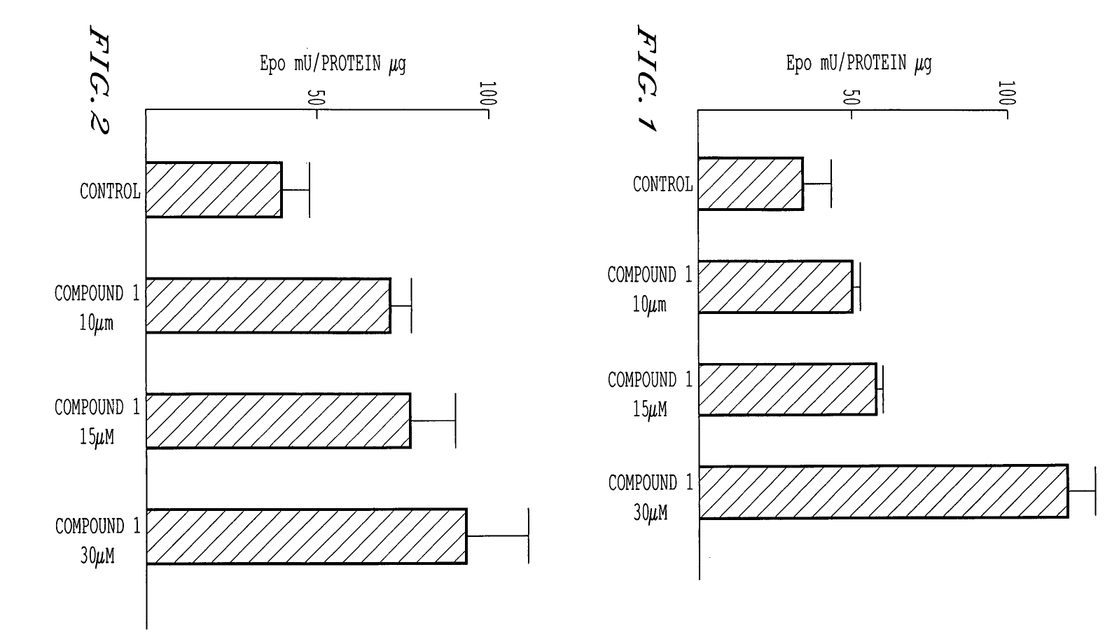

FIG. 2 shows effect of compound 1 on the amount of EPO produced by Hep3B cells at a low O₂ partial pressure. In the case of control samples, the amount of EPO was found to be 39.5±9.1 mU/protein μg. In the cases where compound 1 was added in amounts of 10, 15, and 30 μM, the amount of EPO was found to be 71.0±6.0, 76.0±13.5, and 91.5±17.8 mU/protein μg, respectively; i.e., in these cases as well the amount of EPO increased dose-dependently.

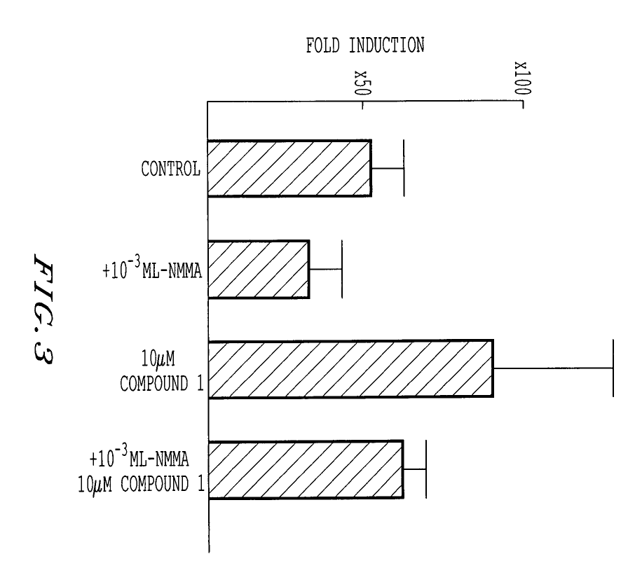

FIG. 3 shows EPO potentiator activity of the wild strain. HI of control samples was found to be 52.2±10.7. When 10⁻³M L-NMMA was added, HI was found to be 32.9±10.6; i.e., EPO potentiator activity was suppressed. When only 10 μM of compound 1 was added, HI was found to be 91.0±36.6, revealing a considerable increase. When 10 μM of compound 1 was added in the presence of 10⁻³M L-NMMA, HI was found to be 63.4±7.5; i.e., suppression of EPO potentiator activity by L-NMMA was canceled.

Modifications and Other Embodiments

Various modifications and variations of the described compositions and methods as well as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the medical, biological, chemical or pharmacological arts or related fields are intended to be within the scope of the following claims.

Incorporation by Reference

Each document, patent application or patent publication cited by or referred to in this disclosure is incorporated by reference in its entirety. Any patent document to which this application claims priority is also incorporated by reference in its entirety. Specifically, priority document U.S. Provision Patent Application No. 60/391,952, filed Jun. 28, 2002 is hereby incorporated by reference.

What is claimed is:

1. A method for treating a pathological condition caused by reduced production of erythropoietin, comprising:
administering to a subject in need thereof an effective amount of one or more of the following compound(s):
N,N′-bis(5 (3,4,5-trimethoxyphenyl)-4-pentenyl)homopiperazine,
an acid-addition salt of N,N′-bis(5-(3,4,5-trimethoxyphenyl)-4 pentenyl)homopiperazine,
a hydrate of N,N′-bis(5-(3,4,5-trimethoxyphenyl)-4-pentenyl)homopiperazine, or
a hydrate of an acid addition salt of N,N′-bis(5-(3,4,5-trimethoxyphenyl)-4 pentenyl)homopiperazine

2. The method according to claim 1, wherein the pathological condition caused by reduced production of erythropoietin is anemia.

3. The method according to claim 1, wherein the pathological condition is chronic anemia.

4. The method according to claim 1, wherein the pathological condition is renal anemia.

5. The method according to claim 1, wherein the pathological condition ishypoplastic anemia.

6. The method according to claim 1, wherein the pathological condition is pure blood cell aplasia.

7. The method of claim 1, comprising administering N,N′-bis(5 (3,4,5-trimethoxyphenyl)-4-pentenyl)homopiperazine.

8. The method of claim 1, comprising administering an acid-addition salt of N,N′-bis(5(3,4,5-trimethoxyphenyl)-4-pentenyl)homopiperazine.

9. The method of claim 1, comprising administering a hydrate of N,N′-bis(5-(3,4,5-trimethoxyphenyl)-4-pentenyl)homopiperazine.

10. The method of claim 1, comprising administering a hydrate of an acid-addition salt of N,N′-bis(5 (3,4,5-trimethoxyphenyl)-4-pentenyl)homopiperazine.

11. The method of claim 1, wherein said administering is done perorally.

12. The method of claim 1, wherein said administering is done parenterally.

13. The method of claim 1, further comprising administering at least one other drug or growth factor.

14. The method of claim 1, further comprising administering erythropoietin.

15. A method for potentiating erythropoietin production comprising
contacting a cell which can produce erythropoietin with an effective amount of one or more of the following compound(s):
N,N′-bis(5-(3,4,5-trimethoxyphenyl)-4 pentenyl)homopiperazine,
an acid-addition salt of N,N′-bis(5-(3,4,5-trimethoxyphenyl)-4 pentenyl)homopiperazine,
a hydrate of N,N′-bis(5-(3,4,5-trimethoxyphenyl)-4-pentenyl)homopiperazine, or
a hydrate of an acid addition salt of N,N′-bis(5-(3,4,5-trimethoxyphenyl)-4 pentenyl)homopiperazine

16. The method of claim 15, wherein said cell is in vivo.

17. The method of claim 15, wherein said cell is in vitro.

18. The method of claim 15, comprising administering said compound to a nonhuman mammal.

19. The method of claim 15, comprising administering said compound to a human.

20. A method for increasing the numbers of red blood cells or megakaryocytes in a subject comprising administering to said subject an effective amount of one or more of the following compound(s):
N,N′-bis(5 (3,4,5-trimethoxyphenyl)-4-pentenyl)homopiperazine,
an acid-addition salt of N,N′-bis(5 (3,4,5-trimethoxyphenyl)-4pentenyl)homopiperazine,
a hydrate of N,N′-bis(5-(3,4,5-trimethoxyphenyl)-4-pentenyl)homopiperazine, or
a hydrate of an acid-addition salt of N,N′-bis(5 (3,4,5-trimethoxyphenyl)-4-pentenyl)homopiperazine.
